(19) United States
(12) Patent Application Publication
(10) Pub. No.: US 2011/0236395 A1
(43) Pub. Date: Sep. 29, 2011

(54) TREATMENT OF FIBROTIC EYE DISORDERS USING AN MMP2 INHIBITOR

(75) Inventors: Ian Michael Wormstone, Norfolk (GB); Lucy Dawes, Norfolk (GB); Julie Ann Eldred, Norfolk (GB); Dylan Edwards, Norfolk (GB); Lisa Hodgkinson, Norfolk (GB)

(73) Assignee: UNIVERSITY OF EAST ANGLIA, (GB)

(21) Appl. No.: 13/063,100

(22) PCT Filed: Sep. 9, 2009
(86) PCT No.: PCT/GB09/51148

§ 371 Date: May 24, 2011

(30) Foreign Application Priority Data
Sep. 9, 2008 (US) 61095554

(51) Int. Cl. A61K 39/395; C07D 209/20; A61K 31/4045; C07C 317/48; A61K 31/195; C07C 323/63; A61K 31/165; C07D 279/12; A61K 31/541; C07D 401/06; A61K 31/454; C07H 21/02; A61K 31/713; C07K 16/40; A61P 27/02; A61P 27/12

(52) U.S. Cl. 424/158.1; 548/496; 514/419; 562/430; 514/562; 564/162; 514/618; 544/58.4; 514/227.8; 546/210; 514/326; 536/24.5; 514/44 A; 530/389.3

(57) ABSTRACT

Inhibitors of matrix metalloproteinase 2 are used to treat or prevent a fibrotic disorder of the posterior capsule of the eye, for example posterior capsule opacification (PCO), or of a tissue or structure of the eye other than the lens or capsular bag.

### FIELD OF THE INVENTION

The present invention relates to the treatment of fibrotic eye disorders, for example TGF-β-mediated fibrotic (including tissue-contraction) disorders of the posterior capsule of the eye and other tissues or structures of the eye, other than the lens or capsular bag, such as the cornea, conjunctiva or sclera, particularly but not exclusively fibrotic complications following eye surgery in humans.

### BACKGROUND OF THE INVENTION

Fibrotic disorders of the eye are common complications arising from surgical treatment of disorders including glaucoma, pterygia and cataract. Many of the underlying mechanisms giving rise to these fibrotic disorders are likely to share common pathways. Further details and discussion of fibrotic complications of glaucoma surgery, cataract or other ocularlens replacement surgery and pterygia surgery are found respectively in Cordeiro, M. F., Prog. Retin. Eye Res. (2002) 21, pages 75-89, Wormstone, I. M., Exp. Eye Res. (2002) 74, pages 337-347, and Di Girolamo, N., et al., Prog. Retin. Eye Res. (March 2004), 23(2), pages 195-228, the contents of all of which are incorporated herein by reference. For example, PCO is the most common complication following cataract or other ocular lens replacement surgery. The condition is caused by regrowth of the lens epithelial cells which, despite the surgeon's best efforts, typically remain on the anterior capsule after the surgery. The cellular regrowth typically invades denuded surfaces of the anterior capsule, the implanted intraocular lens and the previously cell-free posterior capsule. The epithelial cells on the posterior capsule surface give rise to contraction of the tissue matrix, leading to opacification of the posterior capsule and reduction in vision quality.

Transforming growth factor β (TGF-β) has been implicated in a number of fibrotic disorders of the lens, capsular hag, cornea, conjunctiva, sclera and other tissues or structures of the eye. For example, TGF-β has been shown to induce anterior subcapsular cataract (ASC) in a rat lens culture model (Gordon-Thomas, C. et al., Invest. Ophthalmol. Vis. Sci. (1998) 39, pages 1399-1409; Hales, A. M. et al., Invest. Ophthalmol. Vis. Sci. (1995) 36, pages 1709-1713). TGF-β has also been implicated as a causative factor in PCO (Saika, S. et al., Graefes. Arch. Clin. Exp. Ophthalmol. (2000) 238, pages 283-293; Wormstone, I. M. et al., Invest. Ophthalmol. Ns. Sci. (2002) 43, pages 2301-2308). It has been reported that, after trauma (e.g. surgery), active levels of all TGF-β isoforms can be elevated (Ohta, K. et al., Invest. Ophthalmol. Vis. Sci. (2000) 41, pages 2591-2599). The contents of all of these cited publications are incorporated herein by reference.

Trauma to the lens can also modify expression of other factors including matrix metalloproteinases (MMPs), proteases commonly associated with wound healing (Tamiya, S. et al, Exp. Eye Res. (2000) 71, pages 591-597; Wormstone, I. M. et al., Invest. Ophthalmol. Vis. Sci. (2002) 43, pages 2301-2308; the contents of these cited publications are incorporated herein by reference). For example, MMP2 was present in the culture medium of porcine lenses exposed to oxidative stress, but was absent in the medium of clear cultured lenses ((Tamiya, S. et al, Exp. Eye Res. (2000) 71, pages 591-597). Analysis of human lens epithelium also suggested that the most widely studied MMPs—2 and 9—were undetectable using western blot techniques, but the membrane bound MT1-MMP (MMP14) was present (5 mine, A. et al, Curr. Eye Res. (1997) 16, pages 925-929, the contents of which are incorporated herein by reference). This latter protease is associated with the activation of pro-MMP2, but given the absence of MMP2 its functional role in the mechanism of lens disorders is unknown. In particular, it cannot be predicted from this prior art that inhibition of MMP14 would provide a treatment or prevention against PCO or other TGF-β-mediated disorders of the posterior capsule.

It is known that MMP2 and 9 are found in elevated concentrations following the mechanical trauma of sham cataract surgery (Wormstone, I. M., Exp. Eye Res. (2002) 74, pages 337-347). This elevation is relatively transient, but following exposure to TGF-β a sustained increase in MMP 2 and 9 levels in the culture medium is observed. Moreover, capsular bags removed from donors who had previously undergone cataract surgery prior to death also released MMPs in to the medium (Wormstone, I. M., Exp. Eye Res. (2002) 74, pages 337-347). However, since the role of specific MMPs in fibrotic disorders of the eye is undefined, prior to the present invention it could not be predicted that inhibition of them would provide a treatment or prevention against PCO or other TGF-β-mediated disorders of the posterior capsule and other tissues or structures of the eye, other than the lens or capsular bag, such as the cornea, conjunctiva or sclera.

Wong, T. T. et al (Br. I Ophthalmol. (2004) 88, pages 868-872, the contents of which are incorporated herein by reference) have demonstrated an ability of the broad spectrum MMP inhibitor Ilomastat (GM6001) to suppress cell migration onto the posterior capsule surface and thus to suppress consequent capsular bag contraction in the absence of TGF-β. In view of the known role of TGF-β in PCO and other fibrotic disorders of the eye, prior to the present invention it was not apparent that a broad spectrum MMP inhibitor would provide a treatment or prevention in vivo against PCO or other TGF-β-mediated disorders of the posterior capsule and other tissues or structures of the eye, other than the lens or capsular bag, such as the cornea, conjunctiva or sclera.

Dwivedi, D. J. et al (Am. J. Pathol., (2006) 168, pages 69-79, the contents of which are incorporated herein by reference) have reported that both broad-spectrum inhibition of MMPs using GM6001 and allegedly “specific” inhibition of MMP2/9 (using (2R)-[(4-biphenylylsulfonyl)amino]-N-hydroxyl-3-phenylpropionamide) prevents the occurrence of TGF-β-induced anterior subcapsular cataracts (ASCs) in excised rat lenses. However, the inhibitors were used at concentrations (typically micromolar) that were likely to suppress a broad spectrum of MMP family and related members, e.g. the disintegrin and metalloproteinase domain-containing proteases (ADAMs). A recent meeting abstract has reported that ASC formation resulting from TGF-β1 adenoviral overexpression is suppressed in MMP9 knockout transgenic mice (Dwivedi, D. J. Invest. Ophthalmol. Vis. Sci., (2007) 48, E-abstract 4221, the contents of which are incorporated herein by reference). However, this abstract did not study effects on matrix contraction. Prior to the present invention, the role of particular MMP family members in the eye pathologies of interest here remained undefined and therefore was not known to provide potential targets for the treatment or prevention in vivo of PCO or other TGF-β-mediated disorders of the posterior capsule and other tissues or structures of the eye, other than the lens or capsular bag, such as the cornea, conjunctiva or sclera.

Seomun, Y., et al. (Biochem, J. (2001) 358, pages 41-48, the contents of which are incorporated herein by reference) reported that overexpression of MMP2 in the human epithelial lens cell line HLE-B3 can induce expression of α-smooth muscle actin (α-SMA) and asserted that the TGF-β1-induced morphological change in the cells was blocked by an MMP inhibitor ((2R)-2-[(4-biphenylylsulphonyl)amino]-3-phenylpropionic acid). Again, this paper did not study effects on matrix contraction and blocking a purely morphological change in an epithelial lens cell line does not predict treatment or prevention in vivo of PCO or other TGF-β-mediated disorders of the posterior capsule and other tissues or structures of the eye, other than the lens or capsular bag, such as the cornea, conjunctiva or sclera.

Prior to the present invention, therefore, there has been no clear understanding of the role of specific MMPs in TGF-β-mediated cell migration onto the posterior capsule surface or consequent capsular bag contraction, and therefore no treatment thereof has been proposed using specific MMP inhibition, including broader MMP inhibition such that the MMP group includes a specific MMP. Similarly, there has been no clear understanding of the role of specific MMPs in disorders of other tissues or structures of the eye, other than the lens or capsular bag, such as the cornea, conjunctiva or sclera.

There would be considerable advantages in achieving a targeted treatment by inhibition of a specific relevant MMP leaving irrelevant MMPs unaffected, as adverse side effects that may be caused by the unnecessary inhibition of one or more irrelevant MMPs should then be avoided. Furthermore, prior to the present invention there has been no clear understanding of the role of broad spectrum MMP inhibitors in suppression of TGF-β-mediated tissue matrix contraction of the posterior capsule or disorders of other tissues or structures of the eye, other than the lens or capsular bag, such as the cornea, conjunctiva or sclera.

The present invention is based on our surprising finding that inhibitors of matrix metalloproteinase 2 (MMP2) can suppress the said contraction of the posterior capsule and thus provide a treatment or prevention of PCO and other TGF-β-mediated disorders of the posterior capsule as well as disorders, particularly TGF-β-mediated disorders, of other tissues or structures of the eye, other than the lens or capsular bag, such as the cornea, conjunctiva or sclera.

### BRIEF DESCRIPTION OF THE INVENTION

In a first aspect, the present invention provides the use, in the treatment or prevention of a fibrotic disorder of the posterior capsule of the eye, or of a tissue or structure of the eye other than the lens or capsular bag, or in the preparation of a medicament therefor, of an active agent comprising an inhibitor of MMP2.

In a second aspect, the present invention provides an active agent comprising an inhibitor of MMP2, for use in the treatment or prevention of a fibrotic disorder of the posterior capsule of the eye, or of a tissue or structure of the eye other than the lens or capsular bag.

In a third aspect, the present invention provides a composition, for example a pharmaceutical composition (medicament) comprising an effective amount of an active agent comprising an inhibitor of MMP2, and one or more physiologically compatible carrier, diluent or excipient, for use in the treatment or prevention of a fibrotic disorder of the posterior capsule of the eye, or of a tissue or structure of the eye other than the lens or capsular bag.

A particular disorder to be treated or prevented is a TGF-β-mediated disorder of the posterior capsule of the eye, or of a tissue or structure of the eye other than the lens or capsular bag such as the cornea, conjunctiva or sclera. Examples of disorders to be treated or prevented according to the present invention are fibrotic complications of surgery, for example glaucoma surgery (e.g, glaucoma filtration surgery), cataract surgery or other ocular lens replacement surgery, or pterygia surgery, particularly PCO following cataract surgery.

In the various aspects of the present invention, in one embodiment the active agent may, if desired, further comprise at least one other component active agent of the same and/or a different type for treating or preventing a fibrotic disorder of the posterior capsule of the eye, or of a tissue or structure of the eye other than the lens or capsular bag.

The composition may, if desired, contain one or more additional ingredients, which may include effective amounts of one or more components having other physiological activity, suitable amounts of one or more physiologically inert ingredients, or any mixture or combination thereof.

Alternatively, such additional ingredients may be incorporated into other compositions (medicaments), optionally including one or more physiologically compatible carrier, diluent or excipient, for administration separately from the composition according to the present invention, which administration may be prior to, simultaneously with or after the administration of the composition according to the present invention.

The composition according to the present invention may, if desired, consist essentially of the active agent and, if present, the one or more physiologically compatible carrier, diluent or excipient. In this embodiment the active agent may suitably consist essentially of the MMP2 inhibitor.

The composition according to the present invention may, if desired, consist of the active agent and, if present, the one or more physiologically compatible carrier, diluent or excipient. In this embodiment the active agent may suitably consist of the MMP2 inhibitor.

The expressions “inhibitor of MMP2” and “MMP2 inhibitor”, which are interchangeable, used herein include all agents which have the physiological effect of inhibiting the effects of MMP2 in a subject, and therefore include a direct-acting inhibitor of MMP2 in the subject and an indirect-acting inhibitor of MMP2 in the subject. “Inhibition of the effects” of MMP2 includes inhibition of the activation of latent MMP2 as well as inhibition of activated MMP2. The expression “indirect-acting” used herein means that the action is on another substance or group of substances which causes inhibition of the MMP2 in the subject, for example action on a pro-form of MMP2 or an activator of the pro-form of MMP2; when used in connection with other agents the expression shall be understood analogously. An indirect inhibitor of MMP2 includes an agent such as a direct or indirect inhibitor of MMP2 activator and a direct or indirect activator of a direct inhibitor of MMP2. The expressions “inhibition of MMP2”, “MMP2 inhibition” and the like shall be understood analogously.

### DETAILED DESCRIPTION OF THE INVENTION

### MMP Inhibitor

MMP2 is part of the superfamily of proteinases (enzymes), that include the collagenases (MMP1, MMP8, MMP 13), the gelatinases (MMP2, MMP9), the stromelysins (MMP3, MMP10, MMP1), matrilysin (MMP7), metalloelastase (MMP12), enamelysin (MMP 19) and the MT-MMPs (MMP14, MMP15, MMP16, MMP17).

The MMP2 inhibitor may be a specific or non-specific MMP2 inhibitor. A specific MMP2 inhibitor may, for example, be an inhibitor which at the concentration in which it is used is specific to MMP2 alone. A non-specific MMP inhibitor may be a broad spectrum MMP inhibitor. MMP inhibitors which are specific to a group of MMPs within the MMP family, which includes MMP2 and/or activator thereof (e.g. MMP14) and other specific MMPs the inhibition of which does not substantially harm its efficacy according to the present invention, are intended to be covered by the term “MMP2 inhibitor”. For example, an MMP2 inhibitor for use in the present invention may be a gelatinase inhibitor with specificity for at least MMP2.

MMP inhibitors have been reviewed by Whittaker, M. et al., Chemical Reviews (1999) 99, pages 2735-2776. For further information about MMP2 inhibitors, see Arlt M., et al., Cancer Res. (2002), 62, pages 5543-5550; Naglich, J. G. et al., Cancer Res. (2001), 61, pages 8480-5; Nelson, A. R. et al., J. Clin. Oncol. (2000) 18, pages 1135-49; Peterson, J. T., Cardiovasc. Res. (2006), 69, pages 677-87; Sternlicht, M. D., et al., Emerg. Ther. Targets (2000) 4, pages 609-633; Sang, Q-X A et al., Curr. Top, Med. Chem. (2006) 6, pages 289-316; Hu, J. et al., Nat. Rev. Drug Disc. (2007) 6, pages 480-498; and Pirard, B., Drug Disc. Today (2007), 12, pages 640-646. Further examples of MMP inhibitors are disclosed in, for example, the PCT (WO) patent applications WO-A-02/074748, WO-A-02/074749, WO-A-02/074751, WO-A-02/074752, WO-A-02/074767, WO-A-03/040098, WO-A-04/020415, WO-A-05/095362, WO-A-06/004532, WO-A-06/004533, WO-A-06/065215 and WO-A-06/065216. The contents of all these cited documents are incorporated herein by reference.

Specific MMP2 inhibitors include, for example, anti-MMP2 antibodies, inhibitory MMP2-binding fragments of anti-MMP2 antibodies and MMP2-expression inhibiting nucleotides (e.g. MMP2-expression inhibiting small interfering RNA (siRNA)). Inhibitors of activators of specific MMPs include, for example, anti-MMP2-activator antibodies and MMP2-activator-binding fragments thereof, and MMP2-activator-expression inhibiting nucleotides (e.g. MMP2-activator-expression inhibiting siRNA). One example of a specific MMP2 activator is membrane type I MMP (MT1-MMP), an activator which activates a pro-form MMP2 (see Deryugina, E. I, et al., Exp. Cell Res. (February 2001), 263(2), pages 209-223, the contents of which are incorporated herein by reference).

Antibody inhibitors may, for example, be polyclonal or monoclonal antibodies or any combination thereof. Inhibitory binding fragments of such antibodies may, for example, include Fab and F(ab)₂. Such antibodies/fragments are suitably prepared according to standard methods which need no detailed discussion here. Such inhibitors may be specific to more than one MMP, provided that they have specificity for at least MMP2, either directly or indirectly such as via inhibition of an activator thereof.

Suitable MMP2 inhibitors for use in the present invention include, for example, inhibitors of MMP2 and/or one or more of its activators (e.g. MMP14), for example, GM6001 {N-[(2R)-2-(hydroxamidocarbonylmethyl)-4-methylpentanoyl]-L-trypto-phan methylamide}, ((2R)-2-[(4-biphenylylsulphonyl)amino]-3-phenylpropionic acid), (2R)-[(4-biphenylylsulfonyl)amino]-N-hydroxyl-3-phenylpropionamide, Batimastat BB94, BMS275291, CGS27023A/MMI270, Marimastat BB2516, Prinomastat AG3340, Tanomastat/Bay12-9566 and Trocade/Ro32-3555 (for all these see Whittaker M. et al. (1999), Chem. Rev. (1999) 99, pages 2735-2776), Ro 28-2653 and Ro 206-0222 (for these last two see Arlt M., et al., Cancer Res. (2002), 62, pages 5543-5550), MMP-2-expression inhibiting nucleotides such as targeted siRNA knockdown agents against MMP-2, anti-MMP2 antibodies which may, for example be monoclonal or polyclonal, inhibitory MMP-2-binding fragments of such anti-MMP2 antibodies such as Fab or F(ab)_{z}., MMP-14-expression inhibiting nucleotides such as targeted siRNA knockdown agents against MMP-14, anti-MMP14 antibodies which may, for example be monoclonal or polyclonal, inhibitory MMP-14-binding fragments of such anti-MMP14 antibodies such as Fab or F(ab)₂. Other salt forms or free base forms of the above-mentioned MMP-2 inhibitors may also be suitable.

Certain endogenous tissue inhibitors of metalloproteinases (TIMPs) could also be applied to negate MMP2 action, in particular TIMP1, TIMP3 and/or TIMP4.

TIMP2 is implicated in the activation of MMP2, and thus inhibitors of TIMP2 expression or activity are likely to yield therapeutic benefit as active agents or co-agents in the present invention.

### Compositions and Administration Routes

The active agent(s) and composition of the present invention may be administered by any suitable method, including irrigation of the capsular bag during surgery, slow release and/or slowly degradable compositions associated with the lens and/or the capsular bag and/or its vicinity, or administration by direct infusion or injection into the capsular bag or its vicinity.

Conventional composition forms, such as aqueous or oily solutions or suspensions, gels, pastes, emulsions and sterile infusible or injectable aqueous or oily solutions or suspensions may be used.

Irrigation of the capsular bag during surgery (for example, cataract surgery) according to the present invention may suitably be accomplished using a sealed-capsule irrigation device such as the PerfectCapsule® system (Milvella Pty. Ltd., Sydney, Australia) (see Abdelwahab et al, Journal of Cataract and Refractive Surgery, 33(9), 2007, 1619-1623). Such a device also provides a suitable means for introducing a slow release and/or slowly degradable composition in accordance with the present invention into the capsular bag before introduction of the new lens in lens exchange surgery. Alternatively, or in addition, a slow release and/or slowly degradable composition in accordance with the present invention may be injected or infused into the capsular bag behind the new lens during the surgery.

Alternatively, or in addition, a slow release and/or slowly degradable composition in accordance with the present invention may be impregnated into, and/or coated onto, the lens surface of the new intraocular lens inserted during routine cataract surgery, so that the active agent(s) will be gradually released into the capsular bag. The composition may be impregnated into, and/or coated onto, the lens in conventional manner, for example by soaking or spraying.

These composition forms will usually include one or more pharmaceutically acceptable ingredients which may be selected, for example, from adjuvants, carriers, binders, lubricants, diluents, sodium chloride, stabilising agents, buffering agents, emulsifying agents, wetting agents, viscosity-regulating agents, surfactants, preservatives and colorants. As will be understood by those skilled in the art, the most appropriate method of administering the active ingredients is dependent on a number of factors. The compositions for use in the present invention may, if desired, be presented as a dry product for reconstitution with water or other suitable vehicle before use.

For control of release of the active agent(s) according to the present invention a number of effective methods are available. See, for example, Wagh V. D., Inamdar B., Samanta M. K., Polymers used in ocular dosage form and drug delivery systems. Asian J Pharm 2, 2008, 12-17 and the literature references cited therein, the contents of which are incorporated herein by reference. The use of polymers (e.g. cellulose derivatives such as hydroxypropylmethylcellulose (HPMC) and hydroxypropylcellulose (HPC), poly(acrylic acid) (PAA), polyacrylates, cyclodextrins and natural gums, polyorthoesters (POEs) and mucoadhesive polymers, semisolids such as gels, films and other inserts, resins such as ion exchange resins, iontophoretic delivery, gene delivery via naked DNA or vector constructs, and colloidal particles such as microspheres and nanoparticles, may be particularly mentioned. Further discussion of each of these approaches is given in the Wagh paper cited above and the literature references therein, incorporated herein by reference.

In one embodiment of the present invention a number of ingredients are administered via separate pharmaceutical preparations. In this embodiment the different pharmaceutical preparations of active ingredients may be administered simultaneously, sequentially or separately.

Therefore, in one aspect, the present invention provides a kit comprising a preparation of a first active ingredient which is an MMP2 inhibitor, for example a specific or non-specific inhibitor of MMP2, and a preparation of a second active ingredient, and optionally instructions for the simultaneous, sequential or separate administration of the preparations to a patient in need thereof.

The second active ingredient may be selected from a wide range of physiologically active agents, according to the likely or expected symptoms that may need to be relieved in the subject being treated. Such agents may include, for example, an anti-inflammatory agent; a lipid lowering agent such as a statin or a fibrate; a modulator of blood cell morphology such as pentoxyfylline; a thrombolytic or an anticoagulant such as a platelet aggregation inhibitor; a CNS agent such as an antidepressant (such as sertraline); an agent for the treatment of acute or chronic pain, such as a centrally or peripherally-acting analgesic (for example an opioid or derivative thereof), carbamazepine, phenyloin, sodium valproate, amitryptiline or other anti-depressant agent(s), paracetamol, or a non-steroidal anti-inflammatory agent); a parenterally or topically-applied (including inhaled) local anaesthetic agent such as lignocaine or a derivative thereof; or any mixture or combination thereof.

The dosage of active agent(s) to be applied is readily obtained by tests and establishing the dosage is within the skill of the reader, based on the experimental work described below.

### BRIEF DESCRIPTION OF THE DRAWINGS

In the accompanying drawings:

FIG. 1 shows a diagrammatic representation of a post-surgical capsular bag and PCO development;

FIG. 2 shows, in photographic and graphical form, TGF-β-induced matrix contraction with and without the non-specific MMP matrix inhibitor GM6001. Results are shown as (A) photographs of the patches in the patch assay, (B) graphical quantification of the detected area of cell coverage in the patch assay, and (C) graphical quantification of the cell populations in the different patch areas;

FIG. 3 shows the effects of TGF-β2 on the expression of members of the MMP and TIMP families in the human lens cell line FHL-124;

FIG. 4 shows validation of siRNA directed against MMP2 (siMMP2);

FIG. 5 shows the effect of siMMP2 on MMP2 protein expression by zymographic analysis. Results are shown (A) as the band intensity relative to the 24 hour control and (B) as the actual visualised bands in the same order left to right as shown in FIG. 5A;

FIG. 6 shows, in photographic and graphical form, the effect of siMMP2 on TGF-β-induced matrix contraction. Results are shown as (A) photographs of the patches in the patch assay, (B) graphical quantification of the detected area of cell coverage in the patch assay, and (C) graphical quantification of the cell populations in the different patch areas;

FIG. 7 shows the effect of MMP2 neutralising antibodies on TGF-β-induced matrix contraction. Results are shown as (A) photographs of the patches in the patch assay and (B) graphical quantification of the detected area of cell coverage in the patch assay; and

FIG. 8 shows the effect of MMP2 neutralising antibodies on TGF-β-induced wrinkle formation and actin stress fibre formation in human capsular bags. Results are shown as (A) phase contrast micrographs of the capsular bags at the end point (21 days) and (B) stained immunocytochemistry micrographs of the capsular bags at the end point (21 days);

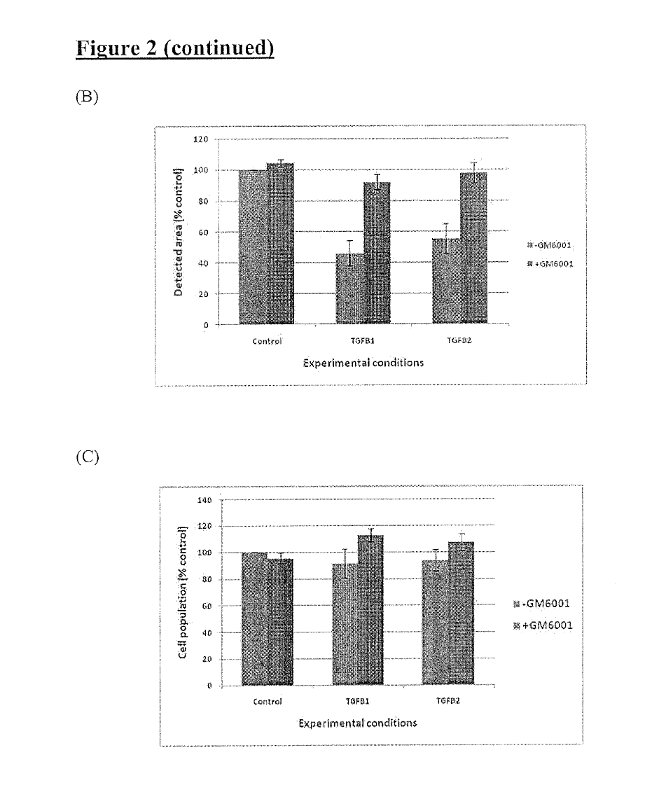

The following legends are helpful when studying FIGS. 2 to 8 of the drawings:

FIG. 2. Broad spectrum inhibition of MMPs prevents TGF-β-induced matrix contraction. FHL 124 cells were seeded to form patches, maintained in serum-free medium before exposure to experimental conditions. All cultures were maintained for 72 hours in 5% EMEM (Eagle's minimal essential medium) alone or 5% FCS (fetal calf serum) EMEM supplemented with 10 ng/mL TGF-β1 or -β2±the MMP inhibitor GM6001 (25 μM). (A) Visual representations of patch assay cultures in different experimental conditions. (B) Quantified data showing differences in patch area. (C) Data showing differences in cell populations between patch areas (Coomassie blue dye was extracted and quantified from the same experiment as presented in (B)). In FIGS. 2(B) and (C) each left hand bar of the bar pairs reports the result in the absence of GM6001 (indicated as “−GM6001” in the key) and the right hand bar reports the result in the presence of GM6001 (indicated as “+GM6001” in the key). Data represent mean±SEM. * Indicates a significant difference from unstimulated control cells. # Indicates a significant difference between siMMP2 treated cells and SCR siRNA counterparts (p≦0.05, ANOVA with the Tukey test).

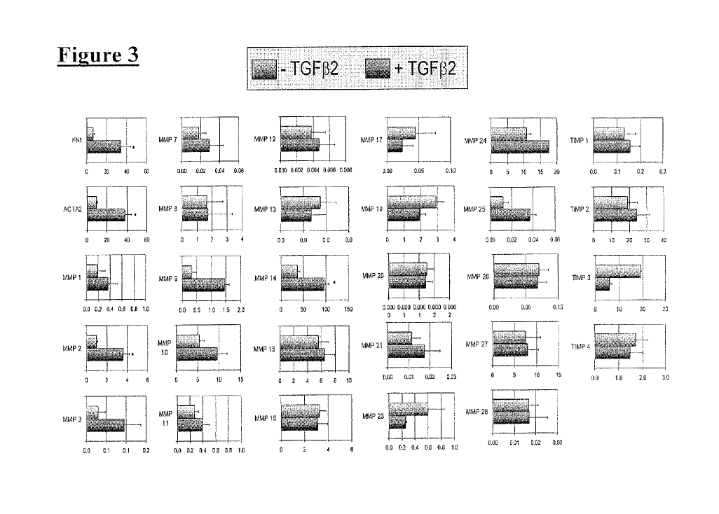

FIG. 3. The effect of 10 ng/ml TGF-β2 on MMP and TIMP gene expression in FHL 124 cells following a 24 hour exposure period. In each case the upper bar reports the result in the absence of TGF-β2 (indicated as “−TGF-β2” in the key) and the lower bar reports the result in the presence of TGF-β2 (indicated as “+TGF-β2” in the key). The x-axis represents the gene of interest/18S expression expressed as mean±SEM, * indicates a significant difference between TGF-β2 treated and control group (p<0.05; student's t-test).

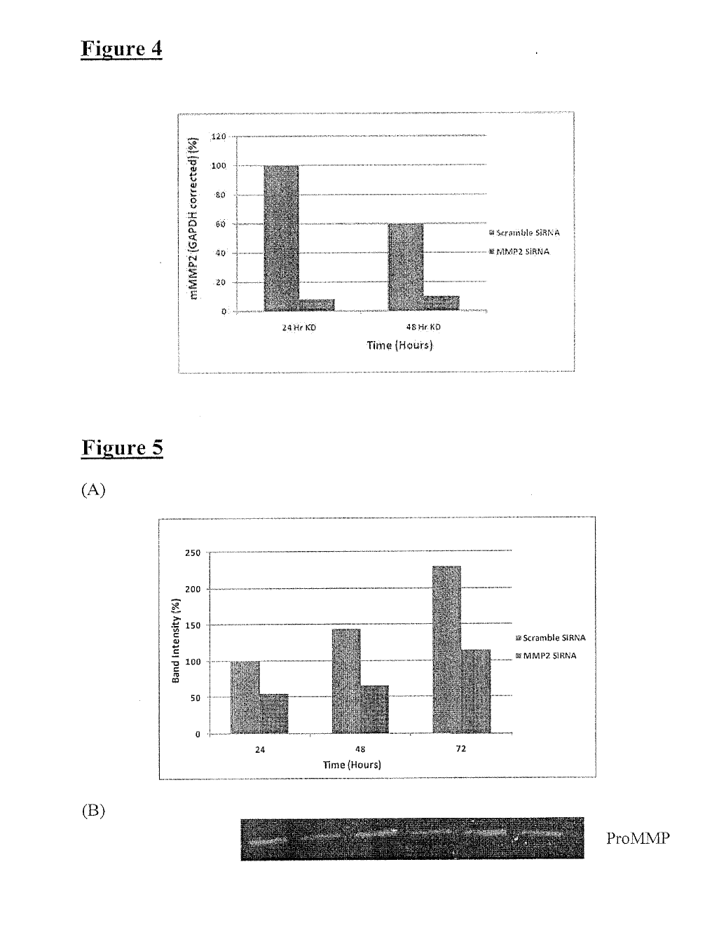

FIG. 4. Validation of siRNA directed against specific inhibition of MMP2 for 24 hours using targeted siRNA techniques leads to a greater than 90% reduction in MMP2 gene expression. Furthermore, after 48 hours from initial MMP2 SiRNA transfection, MMP2 gene expression levels are still reduced by more than 80% in comparison with Scramble SiRNA controls. Each left hand bar of the bar pairs reports the result with Scramble siRNA (indicated as “Scramble SiRNA” in the key) and the right hand bar reports the result in the presence of MMP2 specific siRNA (indicated as “MMP2 SiRNA” in the key).

FIG. 5. SiMMP2 suppresses MMP2 protein expression. Zymographic analysis over a 72 hour time-course shows that MMP2 expression increases in Scramble SiRNA controls and siMMP2-treated cells over time. However, throughout this period the MMP2 level in the siMMP2 treated cells is about 50% of the siRNA control cells (SCR). In FIG. 5A each left hand bar of the bar pairs reports the result with Scramble siRNA (indicated as “Scramble SiRNA” in the key) and the right hand bar reports the result in the presence of MMP2 specific siRNA (indicated as “MMP2 SiRNA” in the key).

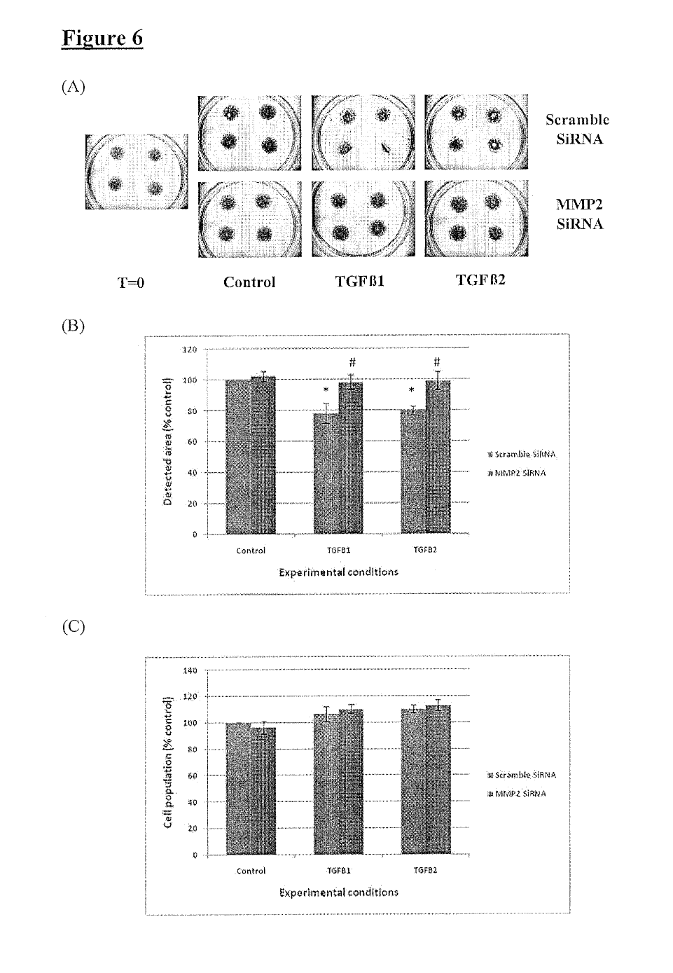

FIG. 6. MMP2 is important for TGF-β-induced matrix contraction. FHL 124 cells were seeded to form patches, then transfected with siRNA targeted to MMP2 or SCR negative control and maintained in Optimem™ (Invitrogen Corporation; Optimem is a modified EMEM, buffered with HEPES and sodium bicarbonate and supplemented with hypoxanthine, thymidine, sodium pyruvate, L-glutamine or Glutamax, trace elements and growth factor; the protein level is minimal (15 μg/mL) with insulin and transferrin being the only protein supplements; phenol red is included as a pH indicator). Patches were measured after 72 hours of culture with 10 ng/mL TGF-β1 or 432. (A) Visual representations of patch assay cultures in different experimental conditions. (B) Quantified data showing differences in patch area. (C) Data showing differences in cell populations between patch areas (Coomassie blue dye was extracted and quantified from the same experiment as presented in (B)). In FIGS. 6(B) and (C) each left hand bar of the bar pairs reports the result with Scramble siRNA (indicated as “Scramble SiRNA” in the key) and the right hand bar reports the result in the presence of MMP2 specific siRNA (indicated as “MMP2 SiRNA” in the key). The data in (B) and (C) represent the mean±SEM (n≦4). *Indicates a significant difference from unstimulated control cells. # Indicates a significant difference between siMMP2 treated cells and SCR siRNA counterparts (p≦0.05, ANOVA with the Tukey test).

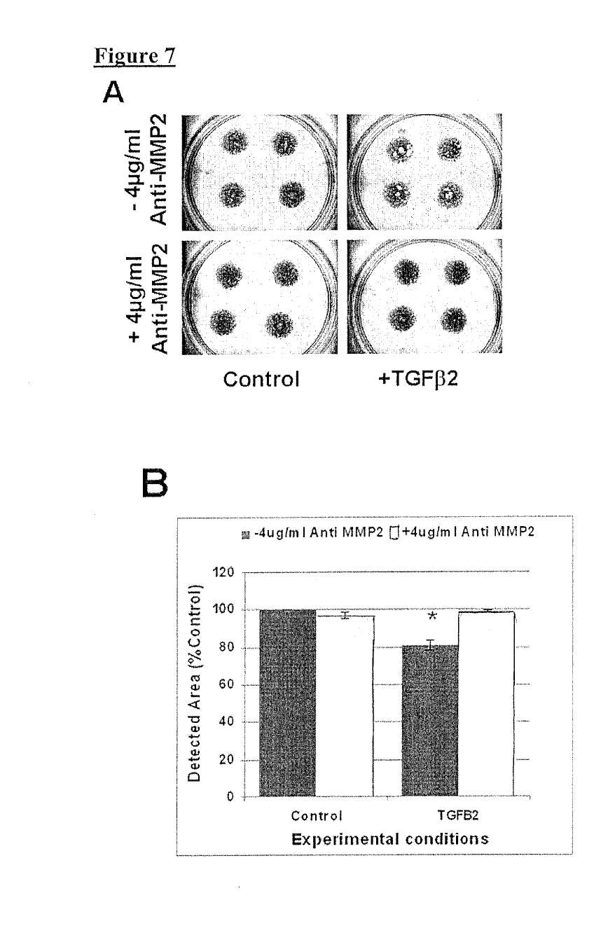

FIG. 7. MMP2 inhibition using neutralising antibody (4 ng/ml) prevents TGF-β2 (10 ng/ml)-induced matrix contraction assessed by patch assay. In FIG. 7(B) each left hand bar of the bar pairs reports the result in the absence of the antibody (indicated as “−4 μg/ml Anti MMP2” in the key) and the right hand bar reports the result in the presence of the antibody (indicated as “+4 μg/ml Anti MMP2” in the key). Data expressed as Mean±SEM (n=3).

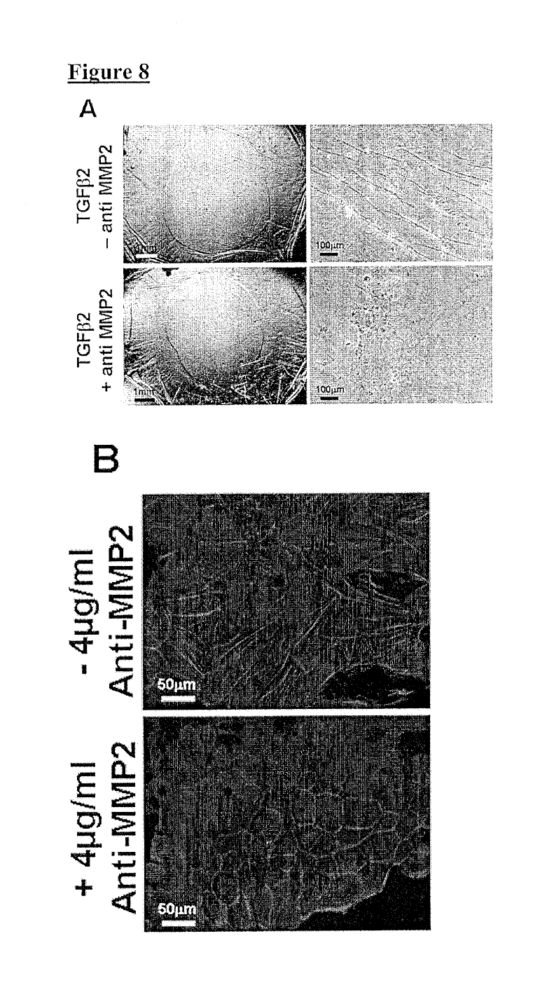

FIG. 8. MMP2 inhibition using neutralising antibody (20 μg/ml) prevents TGF-β2 (10 ng/ml)-induced formation of winkles and stress fibres in human capsular bags (n=3). Images captured at end point (day 21).

### TEST DATA AND DETAILED DESCRIPTION OF THE DRAWINGS

The following non-limiting Test Data are provided for further illustration and explanation of the present invention and the accompanying drawings.

TGF-β is strongly linked with a number of pathologies including posterior capsule opacification (PCO), which develops in a significant number of patients following cataract surgery.

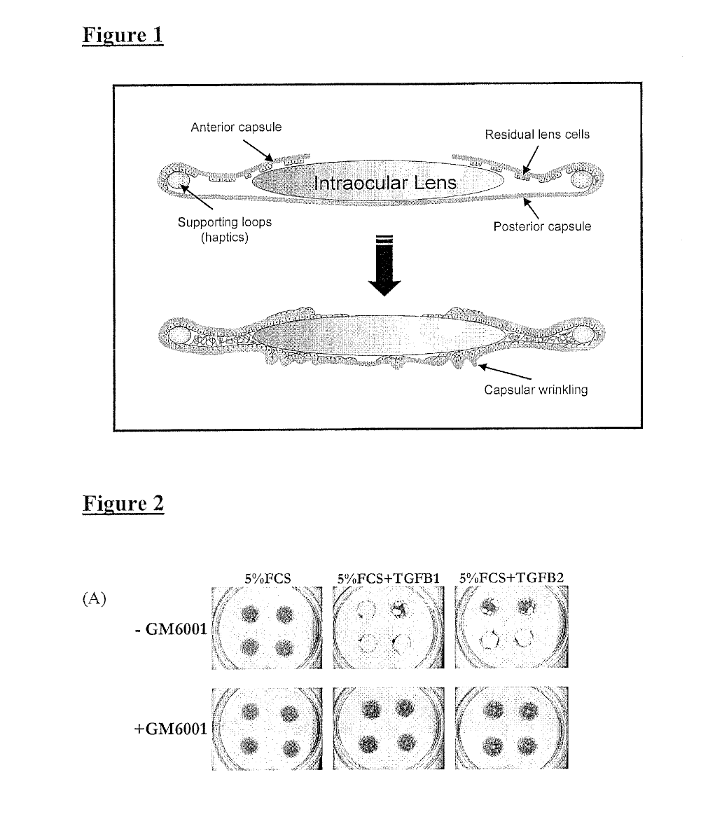

As illustrated diagrammatically in FIG. 1 of the drawings, in PCO, TGF-β causes the matrix on which the cells grow to deform through contraction. This is seen as wrinkling of the lens capsule. When this occurs on the central posterior capsule, the normal uninterrupted path of light becomes disrupted and no longer focuses properly on the retina. Consequently, this affects visual quality of a patient and can necessitate further corrective surgery.

The present invention is based on an important new understanding of the biological mechanisms mediating TGF-β-induced events, which in turn leads to therapeutic targets for the treatment of PCO and other TGF-β-associated conditions.

The data presented in the Figures and discussions here show that MMP2 regulation is critical for TGF-β-mediated matrix contraction. The present invention provides treatment or prevention of fibrotic disorders by using these control systems for MMP2 activity to reduce matrix contraction through reduction in the overall level of MMP2.

### Materials and Methods

The materials and methods are as described above in the legends to each of FIGS. 2 to 6. FHL-124 cells were a gift from Dr John Reddan, Oakland University, Rochester, Mich., USA. TGF-β was sourced from Sigma-Aldrich (also referred to below as “Sigma”), Poole, Dorset, UK. GM6001 was sourced from Calbiochem. siRNA was sourced from Ambion. Sources of other materials are stated below or the materials are easily available. Standard abbreviations are used for routine materials.

Total RNA Extraction and cDNA Generation

Total RNA was extracted from tissue and primary cultured cells (RNeasy micro kits; Qiagen, West Sussex, UK) in accordance with the manufacturer's instructions. In the initial step, RLT buffer (containing-mercaptoethanol) was added directly to phosphate buffered saline (PBS)-washed culture monolayers. Cultured cell lysates were removed with a cell scraper. Each sample was then transferred to an individual Eppendorf tube and passed through a needle and syringe. The remainder of the protocol was as described by the manufacturer and included a DNAse step. Quality control was maintained with an RNA analyzer (Bioanalyzer 2100; Agilent, West Lothian, UK) and an RNA lab chip (6000 Nano lab chip; Agilent) to ensure that 28S and 18S rRNA bands were clearly evident in total RNA samples. RNA was quantified with a spectrophotometer (ND-1000; NanoDrop, Wilmington, Del., USA). For the samples analyzed, the 260:280 ratio ranged from 1.8 to 2.2 (mean, 2.0). Where possible, total RNA was immediately used for cDNA generation or was briefly stored at −80° C. Generation of cDNA was performed with reverse transcriptase (Superscript II; Invitrogen, Paisley, UK) and random primers (Promega, Southampton, UK), according to standard protocols.

### Real-Time PCR

Quantitative real-time PCR was used to analyze mRNA expression for all genes in FHL 124 cells (for a comprehensive description of MMPs and TIMPs primer/probe sets used, see Nuttall R. K. et al. (Mol Cancer Res (2003) 1, pages 333-345. Assuming 100% efficiency in the reverse transcription reaction, either 1 ng or 5 ng cDNA was used in real-time PCR reactions performed using a real-time PCR machine (ABI7700; Applied Biosystems). Reagent-based assays (Taq-Man Universal PCR Master Mix, No AmpErase UNG; Applied Biosystems) with all PCR reagents were used according to the manufacturer's instructions. The amount of amplification associated with priming from genomic DNA contamination was evaluated with control reverse transcription reactions containing all reagents without reverse transcriptase. Conditions for the PCR reaction were 2 minutes at 50° C., 10 minutes at 95° C., and then 40 cycles, each consisting of 15 seconds at 95° C. and 1 minute at 60° C. The cycle number at which amplification entered the exponential phase (cycle threshold [CT]) was determined, and this number was used as an indicator for the amount of target RNA in each sample analyzed. To determine the relative RNA levels in the samples, standard curves for each primer/probe set were prepared by taking cDNA from one sample and making twofold serial dilutions covering the range equivalent to 20 ng to 0.625 ng RNA (for 18S analysis, the range was 1 ng to 0.03125 ng). Differences in the total amount of RNA present in each sample were normalized to endogenous 18S ribosomal RNA gene expression, as previously described (Hodgkinson L. M. et al (Invest. Ophthalmol. Vis. Sci. (2007) 48, pages 4192-99).

### Zymographic Analyses

Gelatinolytic activity of culture media was analyzed by electrophoresis under non-reducing conditions on a 7% SDS-polyacrylamide gel containing 0.5 mg/mL denatured collagen type I (gelatin; Sigma) as previously described by Gavrilovic J. et al. (Cell Regul. (1990) 1, pages 1003-14). After electrophoresis, gels were washed twice in 2.5% Triton-X 100 for 15 minutes and then incubated for 4 hours at 37° C. in 100 mM Tris-HCl (pH 7.9), 30 mM CaCl₂, and 0.02% sodium azide. The gels were stained with Coomassie brilliant blue (Sigma), and the images were captured with a gel scanner.

### Patch Contraction Assay

FHL 124 cells were seeded at four sites on a tissue culture dish at 5000 cells in 25 μl and maintained in EMEM supplemented with 5% FCS until confluent regions spanning about 5 mm developed (Wormstone et al, Exp. Eye Res. (2004), 78, pages 705-14). The medium was then replaced with non-supplemented EMEM, and the cells were cultured for a further 24 hours, followed by the removal of medium from four patch culture dishes fixed for 30 minutes with 4% formaldehyde at room temperature, followed by washing in PBS. The patches were used as a t=0 reference control.

All remaining cell cultures were exposed to experimental conditions for up to 3 days. Experiments were terminated after the appearance of cell-free regions (holes) within the central region of the patch, by fixation for 30 minutes with 4% formaldehyde at room temperature. The cells were washed in PBS (Sigma-Aldrich, Poole, UK) and stained with Coomassie brilliant blue (a total protein dye) for 30 minutes, to enable patches to be visualized and measured. The cells were washed several times in PBS to remove excess dye.

Images of patches were captured on a CCD camera using grabber software (Synoptics, Cambridge, UK) and analyzed (PC Image; Foster Findlay, Newcastle-upon-Tyne, UK). After the patches had been measured, PBS medium was aspirated from the culture dish and replaced with 1 ml of 70% ethanol allowing Coomassie blue dye within the cells to be dissolved. The culture dishes were placed on a rotary shaker for 1 hour until all the dye had been extracted from the cells. A 200-μl sample of dye from each dish was placed in a clear plastic 96-well microtiter plate and the absorbance read at 550 nm with a multilabel counter (Wallac Victor 2, model 1420; with Workout version 15 software, Perkin Elmer Optoelectronics, Cambridge, UK). The principle of using dye content (i.e., total protein) has been reported to be proportional to the number of cells (Knott R M et al, Curr Eye Res. (1998) 17, pages 1-8).

### SiRNA Transfection

Custom made MMP2 siRNA: sense 5′-GGACACACUAAAGAAGAUGTT-3′ (SEQ. ID. NO: 1), antisense 5′-CAUCUUCUUUAGUGUGUCCTT-3′ (SEQ. ID. NO: 2) and siRNA negative control (universal scrambled siRNA) were used. Both siRNAs were purchased from Ambion Ltd. (Huntingdon, UK). FHL 124 cells were seeded onto 35-mm culture dishes at either 25,000 cells in 1.5 ml for protein extraction or as four patches of 5000 cells, for patch assay analysis. Cells were maintained in EMEM supplemented with 5% FCS for 3 days and then serum starved for 1 day. Transfections were performed with 100 nM siRNAs according to the manufacturer's instructions. Briefly, 1 μl MMP2 siRNA or siRNA negative control (final concentration of 100 nM) was added to 184 μl reduced-serum medium (Optimem™, Invitrogen). In addition, 5 μl oligofectamine (Invitrogen) was added to 10 μl of the Optimem medium. The two solutions were incubated at room temperature for 5 minutes and then mixed by gentle agitation and incubated at room temperature for a further 15 to 20 minutes. Meanwhile, the serum-free medium was aspirated from the cell preparations and replaced with 2 ml of the reduced-serum Optimem medium. This solution was aspirated and replaced with 800 μl of fresh reduced-serum Optimem medium. After the incubation period, 200 μl of siRNA transfection mix was added to the cell preparations. The cells were incubated at 35° C. in a 5% CO₂ atmosphere for 4 hours, to initiate transfection. Following this period a further 500 μl of Optimem was added to the culture dishes and maintained for a further 20 hours prior to addition of experimental agents.

### Antibodies

MMP2 neutralising antibody was obtained from Millipore (USA).

### Human Capsular Bag Experiments

Sham cataract operations were performed on human donor lenses. Experiments were carried out on a match-paired basis, such that preparations were treated with 10 ng/ml TGF-β2±20 μg/ml MMP2 antibody. Ongoing observations were by phase contrast microscopy and at end point (t=21 days) immunocytochemistry was performed.

### Results

### The Broad Spectrum MMP Inhibitor GM6001 Inhibits TGF-β-Induced Contraction

Treatment with either 10 ng/ml TGF-β1 or TGF-β2 resulted in a significant contractile response, such that cell/matrix free areas appeared and the patch area was reduced relative to untreated controls following 72 hours in experimental conditions (FIGS. 2A and B). The presence of 25nM GM6001 (a broad spectrum MMP inhibitor) alone had no significant effect on patch area relative to the control 5% FCS EMEM group (FIGS. 2A and B). Addition of 25 μM GM6001 in the presence of 10 ng/ml TGF-β1 or TGF-β2 suppressed the formation of cell free zones and the patch area did not significantly differ from the un-stimulated control group (FIGS. 2A and B). Estimation of total protein content by Coomassie blue dye extraction from these patches showed no significant changes with any experimental group, indicating similar cell populations (FIG. 2C). The inhibitory actions of GM6001 suggest a likely role for MMPs in TGF-β-mediated events. However, one cannot identify from this data which specific MMP family members are important.

### Gene Expression Profile of MMP Family Members in Response to TGF-β2

A screen approach for TGF-β-responsive MMP family genes was carried out using real-time PCR. Following a 24 hour exposure of the human lens cell line FHL 124 to 10 ng/ml TGF-β2, changes in gene expression were determined relative to non-treated control cells (FIG. 3).

Quantitative analysis was obtained by expressing the data of the gene of interest normalized with respect to the level of 18S message, which serves as a house-keeping control gene. Using this method, it was apparent that the majority of MMP gene expression was not significantly affected by exposure to 10 ng/ml TGF-β2. However, two genes—MMP2 and MMP14—were seen to significantly increase.

It is noted that three additional family members; following TGF-β exposure, did show expression greater than 2-fold that of un-stimulated control cells; however, this change was not statistically significant. In order to validate the TGF-β response in the cells, established TGF-β inducible genes (αSMA(ACTA2) and fibronectin) were also tested; a significant increase in expression was observed.

Inhibition of MMP2 Expression by Targeted siRNA

From the real-time PCR analysis, MMP2 was identified as a likely mediator of TGF-β-induced contraction. Therefore, this specific gene was inhibited using siRNA methods. QRT-PCR was used to validate siRNA against MMP2 in FHL 124 cells after 24 hours in transfection conditions. MMP2 gene expression was significantly inhibited, by 90% relative to non-stimulated negative control, and following 48 hours in transfection conditions inhibition still remained >70% (FIG. 4). To determine whether the inhibition at the message level was also observed at the protein level, gelatin zymography was used. Inhibition of MMP2 expression was again observed, such that levels were approximately 50% of the Scrambled siRNA control group over a 72 hour period (FIGS. 5A and B).

Having demonstrated a tool to specifically inhibit MMP2, the role of MMP2 in TGF-β-mediated contraction was determined. Patches transfected with siRNA against MMP2 did not show a statistical difference in patch area from the SCR control when maintained in non-stimulated conditions (i.e., no TGF-β; FIG. 6). Addition of 10 ng/ml, TGF-β1 or -β2 induced a significant reduction in patch area compared with non-stimulated SCR controls at the 72-hour time point (FIG. 6). Patches transfected with siMMP2 treated with TGF-β1 or TGF-β2 did not show any evidence of contraction and area did not differ significantly from non-TGF-β-treated controls.

### MMP2 Neutralising Antibody Inhibits TGF-β-Induced Contraction

Specific MMP2 inhibition using MMP2 neutralising antibody (4 ng/ml) prevented TGF-β2-induced matrix contraction in FHL-124 cells (FIG. 7).

### MMP2 Neutralising Antibody Inhibits TGF-β-Induced Wrinkling of the Capsular Bag

Human capsular bags exposed to 10 ng/ml TGF-β2 exhibited marked contraction/wrinkling of the posterior capsule. Co-addition of MMP2 neutralising antibody (20 μg/ml) significantly reduced wrinkle formation (FIG. 8).

### DISCUSSION AND CONCLUSIONS

The work underlying the present invention identifies which of the MMP family members are TGF-β inducible and thus could contribute to the contractile response. Of the 23 MMP family members studied, only MMP2 and MMP14 were statistically significantly increased (FIG. 3). Since specific inhibition of both MMP2 and MMP14 is achievable, such inhibition provides a novel route for the treatment and prevention fibrotic disorders of the posterior capsule of the eye, for example PCO, or of a tissue or structure of the eye other than the lens or capsular bag. Using siRNA to provide targeted knockdown, MMP2 was found to play a key role in contraction, but MMP14 was not.

The mechanism by which MMP2 or MMP14 could influence events, and hence the mechanism by which inhibition of these MMPs may prevent or treat PCO, is of great interest. It has recently been shown that MMP2 can release fibroblast growth factor 2 (FGF-2) from the lens capsule (Tholozan, F. M., et al, Mol. Biol. Cell (2007), 18, pages 4222-4231, the contents of which are incorporated herein by reference). It is possible that other matrix binding growth factors such as TGF-β could also be regulated in this manner. Interestingly, FGF is also known to exacerbate TGF-β-induced changes (Cerra, A. et al, Mol. Vis. (2003), 9, pages 689-700, the contents of which are incorporated herein by reference). Therefore, it could be speculated that increased MMP production/secretion in response to TGF-β could release FGF from the capsule, which in turn enhances a wound-healing response and PCO formation.

Without wishing to be bound by any particular theory of operation of the invention, we tentatively propose that the concept of a “contributory MMP”, as introduced earlier in this description, may be related to the ability of the MMP to promote release of one or more growth factor, particularly one or more matrix binding growth factor such as FGF (e.g. FGF-2) from the lens capsule or other tissue structure of the eye.

The foregoing describes the present invention broadly and without limitation to particular embodiments. Variations and modifications as will be readily apparent to those skilled in the art are intended to be included in the scope of the application and subsequent patents.

1. Use, in a method for the treatment or prevention of a fibrotic disorder of the posterior capsule of the eye, or of a tissue or structure of the eye other than the lens or capsular bag, or in the preparation of a medicament therefor, of an active agent comprising an inhibitor of MMP2.

2. An active agent comprising an inhibitor of MMP2, for use in the treatment or prevention of a fibrotic disorder of the posterior capsule of the eye, or of a tissue or structure of the eye other than the lens or capsular bag.

3. A composition, for example a pharmaceutical composition (medicament), comprising an effective amount of an active agent comprising an inhibitor of MMP2, and one or more physiologically compatible carrier, diluent or excipient, for use in the treatment or prevention of a fibrotic disorder of the posterior capsule of the eye, or of a tissue or structure of the eye other than the lens or capsular bag.

4. An agent according to claim 2, wherein the MMP2 inhibitor has inhibiting activity against MMP2 and/or its activators (for example MMP14) and optionally one or more other MMPs.

5. An agent according to claim 2, wherein the MMP2 inhibitor is selected from GM6001 {N-[(2R)-2-(hydroxamidocarbonylmethyl)-4-methylpentanoyl]-L-tryptophan methylamide}, ((2R)-2-[(4-biphenylylsulphonyl)amino]-3-phenylpropionic acid), (2R)-[(4-biphenylylsulfonyl)amino]-N-hydroxyl-3-phenylpropionamide, Batimastat BB94, BMS275291, CGS27023A/MMI270, Marimastat BB2516, Prinomastat AG3340, Tanomastat/Bay12-9566, Trocade/Ro32-3555, Ro 28-2653, Ro 206-0222, TIMP1, TIMP3, TIMP4, MMP-2-expression inhibiting nucleotides such as targeted siRNA knockdown agents against MMP-2, for example SEQ. ID. NOs: 1 and 2, anti-MMP2 antibodies, inhibitory MMP-2-binding fragments of such anti-MMP2 antibodies, MMP-14-expression inhibiting nucleotides such as targeted siRNA knockdown agents against MMP-14, anti-MMP14 antibodies, inhibitory MMP-14-binding fragments of such anti-MMP14 antibodies, and inhibitors of TIMP2 expression or activity.

6. An agent according to claim 2, wherein the MMP2 inhibitor is a specific inhibitor of one or both of MMP2 and MMP14 and is used at a concentration where the said inhibition is specific to one or both of MMP2 and MMP14, whereby in use the activity of MMP2 is inhibited.

7. A composition according to claim 3, which is a slow release and/or slowly degradable composition.

8. A composition according to claim 3 for the treatment or prevention of a fibrotic disorder of the eye by (a) contacting the compositions (e.g. by topical administration or irrigation) with a target tissue or structure (i.e. a tissue or structure to which the active agent needs to be applied according to the present invention, for example a site at which fibrosis is to be prevented), or by administration by direct infusion or injection to a target tissue or structure, suitably during surgery, (b) slow release and/or slowly degradable compositions associated with a target tissue or structure or its vicinity, or (c) administration by direct infusion or injection to a target tissue or structure or its vicinity.

9. A composition according to claim 8, wherein: for treatment or prevention of fibrotic complications of glaucoma filtration surgery, the target tissue or structure is the sclera; for treatment or prevention of fibrotic complications of cataract surgery, for example PCO, the target tissue or structure is the capsular bag; or for treatment or prevention of fibrotic complications of pterygia surgery, the target tissue or structure is the conjunctiva or cornea.

10. A composition according to claim 3, for use in ocular lens replacement surgery by irrigation and/or by association with the lens and/or the capsular bag and/or its vicinity, or by administration by direct infusion or injection into the capsular bag and/or its vicinity.

11. An intraocular lens for use in ocular lens replacement surgery, wherein the lens has associated with it an active agent comprising an MMP2 inhibitor, for the treatment or prevention of a fibrotic disorder of the posterior capsule of the eye.

12. A lens according to claim 11, wherein the MMP2 inhibitor has inhibiting activity against MMP2 and/or its activators (for example MMP14) and optionally one or more other MMPs or is present in a composition comprising an effective amount of an active agent comprising an inhibitor of MMP2, and one or more physiologically compatible carrier, diluent or excipient.

13. A kit for use in the treatment or prevention of a fibrotic disorder of the posterior capsule of the eye, or of a tissue or structure of the eye other than the lens or capsular bag, comprising a preparation of a first active ingredient which is an MMP2 inhibitor and a preparation of a second active ingredient, and optionally instructions for the simultaneous, sequential or separate administration of the preparations to a patient in need thereof.

14. A kit according to claim 13, wherein the MMP2 inhibitor has inhibiting activity against MMP2 and/or its activators (for example MMP14) and optionally one or more other MMPs or is present in a composition comprising an effective amount of an active agent comprising an inhibitor of MMP2, and one or more physiologically compatible carrier, diluent or excipient.

15. A lens according to claim 11, wherein the fibrotic disorder of the eye is a TGF-β-mediated disorder.

16. A lens according to claim 11, wherein the fibrotic disorder of the eye is PCO or other complication of cataract surgery.

17. A lens according to any on claim 11, wherein the fibrotic disorder of the eye is a complication of glaucoma surgery.

18. A lens according to claim 11, wherein the fibrotic disorder of the eye is a complication of pterygia surgery.


SEQUENCE LISTING (2 sequences)

SEQ ID NO: 1 - DNA, 21 bp, Artificial
  MMP2 siRNA sense
ggacacacuaaagaagaugtt

SEQ ID NO: 2 - DNA, 21 bp, Artificial
  MMP2 siRNA antisense
caucuucuuuagugugucctt